(19) United States
(12) Patent Application Publication
(10) Pub. No.: US 2021/0313040 A9
(43) Pub. Date: Oct. 7, 2021

(54) EXPERT-DRIVEN, TECHNOLOGY-FACILITATED INTERVENTION SYSTEM FOR IMPROVING INTERPERSONAL RELATIONSHIPS

(71) Applicant: UNIVERSITY OF SOUTHERN CALIFORNIA, LOS ANGELES, CA (US)

(72) Inventors: GAYLA MARGOLIN, LOS ANGELES, CA (US); ADELA C. AHLE, SAN FRANCISCO, CA (US); MATTHEW WILLIAM AHLE, SAN FRANCISCO, CA (US); THEODORA CHASPARI, COLLEGE STATION, TX (US); SHRIKANTH SAMBASIVAN NARAYANAN, SANTA MONICA, CA (US)

(21) Appl. No.: 16/501,128

(22) Filed: Mar. 2, 2018

Related U.S. Application Data
(60) Provisional application No. 62/563,192, filed on Sep. 26, 2017.

(51) Int. Cl. G16H 20/70; G16H 50/20; G06Q 50/00; G16H 40/67; G16H 50/70; G09B 19/00; H04M 1/725; A61B 5/00; A61B 5/16

(52) U.S. Cl. CPC G16H 20/70; G16H 50/20; G06Q 50/01; G16H 40/67; G16H 50/70; A61B 5/021; H04M 1/725; A61B 5/486; A61B 5/165; A61B 5/7267; G09B 19/00

(57) ABSTRACT

A method for promoting interpersonal interactions includes a step of receiving data streams from a plurality of mobile smart devices from a plurality of users, the data streams recording information about users' daily lives. Intervention signals are sent to a user in response to data acquired from two or more individuals and interpreted with respect to user internal states, moods, emotions, predetermined behaviors, and interactions with other users.

### STATEMENT REGARDING FEDERALLY SPONSORED RESEARCH OR DEVELOPMENT

The invention was made with Government support under Contract No. R21 HD072170-A1 awarded by the National Institutes of Health/National Institute of Child Health and Human Development; Contract Nos. BCS-1627272, DGE-0937362, and CCF-1029373 awarded by the National Science Foundation; and Contract No. UL1TR000130 awarded by the National Institutes of Health. The Government has certain rights to the invention.

### TECHNICAL FIELD

In at least one aspect, the present invention provides a novel, automatic framework for the development and evaluation of mobile, adaptive interventions used to improve interpersonal relationships. In particular, through the integration of expert-knowledge and automated, data-driven methods, this technology-facilitated framework monitors, measures, and quantifies signal-derived human information and provides prompts, suggestions, and support to elicit behavioral change.

### BACKGROUND

Interpersonal relationships refer to acquaintances, close bonds, and affiliations between two or more people across personal, business, educational, and social domains. The quality of these interpersonal relationships is crucial for people's quality of life, well-being, and health. Strained personal and family relationships have been extensively linked to a variety of negative outcomes, including psychological disorders and physical health problems across the lifespan (Burman & Margolin, 1992; Coan, Schaefer, & Davidson, 2006; Grewen, Andersen, Girdler, & Light, 2003; Springer, Sheridan, Kuo, & Carnes, 2007; Holt-Lunstad, Smith, & Layton, 2010; Leach, Butterworth, Olesen, & Mackinnon, 2013; Robles & Kiecolt Glaser, 2003). Similarly, problems in professional relationships have been associated with reduced productivity and decreased well-being (Lawler, 2010; Sacker, 2013; Sonnentag, Unger, & Nagel, 2013).

Current interventions aiming to improve relationship functioning largely rely on participants' retrospective self-reports of their relationship functioning and therapists' observations of their interaction quality. While these are valuable sources of information, traditional therapy interventions have shown only moderate effectiveness in clinical trials (e.g., Lunbald & Hansson, 2005); treatment efficacy may in part be limited by the inherently subjective nature of human judgment; moreover, these interventions cannot provide in-the-moment feedback when problems actually occur in people's day-to-day lives. Additionally, traditional therapies reach only a fraction of individuals who are experiencing significant relationship problems and related difficulties (Mayberry, Nicewander, Qin, & Ballard, 2006). Emerging technological advances now make it possible to monitor people outside the laboratory and collect real-life data about their behavior, interactions, and mental state, and felt-sense. The valuable information about interpersonal dynamics embedded in this multimodal data is thus useful for creating novel, automated and semi-automated intervention systems tailored to individuals to improve their relationships. Such intervention systems rely on human knowledge provided by life-sciences experts accompanied by data-scientific solutions that are able to enhance and complement the human-guided suggestions. In this way, technology can increase people's awareness of emotions, feelings, and problematic behaviors when they occur, provide warnings before problems or conflicts develop, and identify positive and negative interpersonally-relevant states and events beyond what can be identified through traditional therapy. Therapists, on the other hand, can obtain quantitative feedback on their clients' behavior and progress and can adjust interventions with data-driven solutions. These techniques could, therefore, improve individual mental and physical health, democratize access to mental health care, and contribute to saved revenue over time.

Beyond traditional office-based therapy, current online interventions widely rely on web-based educational materials and questionnaires to improve and support interpersonal relationships (Doss, Bensen, Georgia, & Christensen, 2013; Larson et al., 2007). These strategies are widely accessible and can provide initial feedback on relationship quality, but are not highly detailed, do not provide in-the-moment monitoring, feedback, and intervention, and cannot be easily personalized. Other interventions involve remote, online conference sessions with experts (Ianakieva et al., 2016). While these can be effective, they are impossible to scale in large and underprivileged populations, since the presence of experts is costly and not always guaranteed. One potential avenue to increase access to and the effectiveness of interpersonal interventions is to use ambulatory technology that can understand people's behavior, emotions, and felt-sense and provide automated suggestions for positive changes. Recent interdisciplinary studies have examined the possibility of real-life ambulatory monitoring to capture well-being indices and track the progress of mental health conditions and corresponding therapies (Hung, & Englebienne, 2013; Lane et al., 2014; Gideon et al., 2016). However, these studies have focused solely on individual-level functioning, with no previous work, to the best of our knowledge, attempting to monitor and improve social dynamics and interpersonal relations in groups of people.

Accordingly, there is a need for improved methods and systems for monitoring and improving interpersonal relationships.

### SUMMARY

The present invention solves one or more problems of the prior art by providing in at least one embodiment, a system that involves the development, tracking, and evaluation of data-driven interventions through a technology-support system that integrates prior knowledge of human-experts, processes multimodal information acquired from a group of people, and uses data-science, machine learning, and automatic control-based methodologies to create individualized suggestions for altering daily patterns and dynamics of interpersonal relationships (e.g., predict and prevent conflict episodes, increase the frequency of positive interactions, support relationship bonding, aid in expressing viewpoints or emotions in an adaptive manner, effectively problem-solve relationship issues, improve conflict resolution strategies, resolve conflict or restore relationship functioning after conflict has occurred). This system has applications for a variety of relationships (e.g. couples, friends, families, co-workers) and can be employed by individuals or implemented on a broad scale by institutions and large interpersonal networks (e.g. hospitals, military settings).

In the context of the present invention, passive, mobile, ambulatory technologies have been employed to monitor couple dynamics in real-life. Through appropriately designed signal processing and machine learning techniques, phenomena of interest that can affect the quality of interpersonal relationships can be detected, such as the occurrence of conflict. This study was published in IEEE Computer (Timmons et al., 2017) and received attention from the US (e.g. NBC, Daily Mail), international (e.g. Frankfurter Allgemeine Sonntagszeitung, Sabato) and technology- and science-focused (e.g. Digital Trends, Tech Crunch, Tech News Expert, Science Newsline) media (see News Coverage section); the entire disclosures of these publications is hereby incorporated by reference.

### BRIEF DESCRIPTION OF THE DRAWINGS

FIG. 1 is a schematic of a framework implementing embodiments of the invention.

FIG. 2 is a schematic of a smart device used in the system of FIG. 1.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

In an embodiment, a method for monitoring interpersonal relationships is provided. The method includes a step of receiving data streams from a plurality of mobile smart devices from a plurality of users. The data streams record information about users' daily lives. Intervention signals are sent to a user in response to data acquired from two or more individuals and interpreted with respect to user internal states, moods, emotions, predetermined behaviors, and interactions with other users.

In a refinement, the intervention signals are determined by algorithmic signal processing and/or machine learning solutions such that the intervention signals are responsive, interactive, and adaptive to the users.

In a variation, the method further includes a step of incorporating human expert knowledge into a determination of the intervention signals. The human expert knowledge is integrated and includes prompts sent at random intervals and/or according to specific time schedules. In a refinement, reminders designed to help users reach their daily goals can be sent. The reminders can include spending a certain amount of time together, achieving a certain ratio of positive to negative interactions, or having a certain amount of physical contact.

In another variation, the sending of interventions can be triggered by algorithms that automatically detect and predict moods and events to send prompts to oneself or to other users in a social network. The interventions can also include sending prompts after events of interest have occurred. The moods and events can include risky behaviors, extreme emotions, and/or negative moods. Further, the prompts can include warning people that conflict or other events are likely to occur, prompting people to engage in relaxation exercises, take a break, give a compliment, or to do something nice for someone else. In a refinement, the prompts can instruct users to reflect on an occurrence of an event, engage in relationship building activities, initiate positive contact, or discuss a topic together.

In another variation, the method includes a step of providing feedback to the users to encourage beneficial aspects of interpersonal relationships. To provide feedback, expert-knowledge can be applied with personal and interpersonal information captured from human monitoring systems integrated through signal processing, data-scientific, and machine learning solutions. Further, a human state can be recognized, understood, and predicted from this information and actionable feedback can provided to improve it in relation to corresponding relationship functioning. Measurable indices of individual and interpersonal behavior consisting of input for closed-loop systems can automatically provide suggestions towards a desired state.

In yet another variation, heuristic, machine-learning, or control-theoretical approaches are applied and can be automatically trained, tuned, and/or perturbed towards optimizing a desired criterion to minimize conflict and maximize positive interactions. A model can be constructed for interpersonal dynamics that occur when a set of individuals linked through a relationship interacts with each other and with their environment. The method can further include a step of learning each other's patterns over time so that accuracy and effectiveness of interventions increase with use.

In still another variation, the method includes a step of investigating an impact of each prompt and intervention on individual and interpersonal functioning and providing feedback about which interventions ate most helpful population-wide and which are better for specific users, couples, or groups of users. The intervention schemes can be performed quantitatively through signal- and data-derived measures indicative of individual characteristics and relationship functioning concepts.

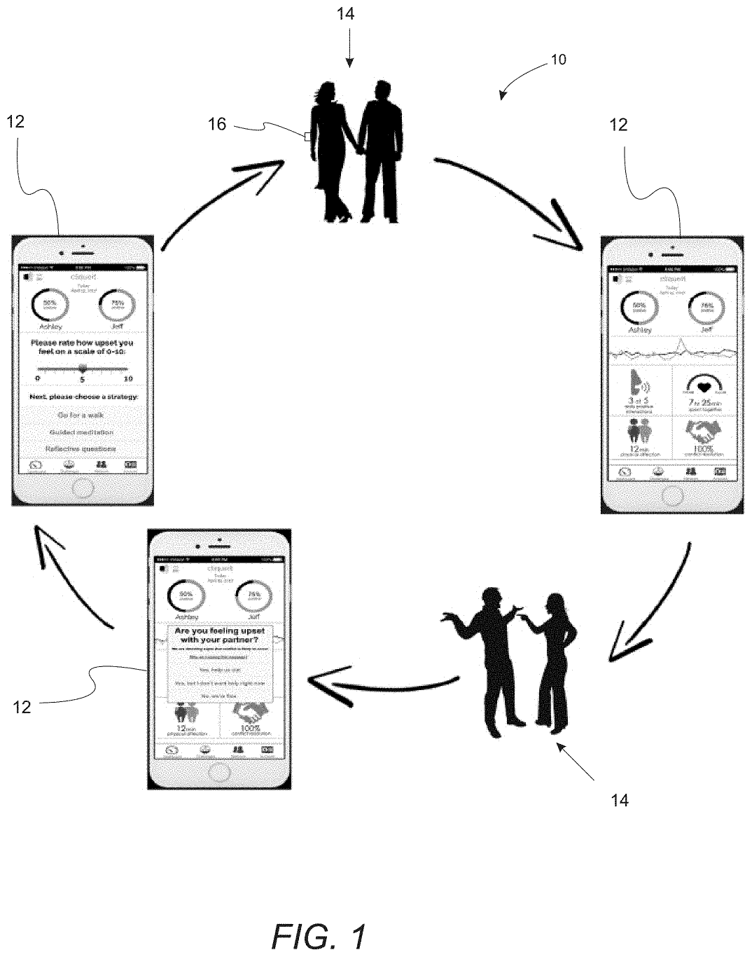

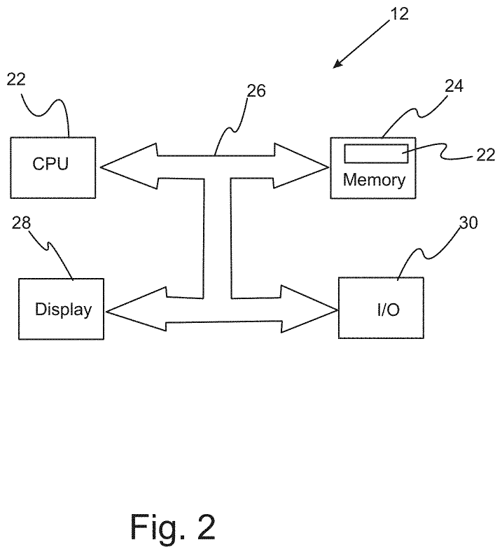

In an embodiment, a system that implements the previously described methods is provided. With reference to FIGS. 1 and 2, the system 10 includes a plurality of mobile smart devices 12 operated by a plurality of users 14. In a variation, the system further includes a plurality of sensors 16 (e.g., heart rate sensors, blood pressure sensors, etc.) worn by the plurality of users. The plurality of mobile smart devices 12 used this system typically includes a microprocessor and a non-volatile memory on which instructions for implementing the method are stored. For example, smart devices 12 can include computer processor 22 that executes one, several, or all of the steps of the method. It should be appreciated that virtually any type of computer processor may be used, including microprocessors, multicore processors, and the like. The steps of the method typically are stored in computer memory 24 and accessed by computer processor 22 via connection system 26. In a variation, connection system 26 includes a data bus. In a refinement, computer memory 24 includes a computer-readable medium which can be any non-transitory (e. g., tangible) medium that participates in providing data that may be read by a computer. Specific examples for computer memory 24 include, but are not limited to, random access memory (RAM), read only memory (ROM), hard drives, optical drives, removable media (e.g. compact disks (CDs), DVD, flash drives, memory cards, etc.), and the like, and combinations thereof. In another refinement, computer processor 22 receives instructions from computer memory 24 and executes these instructions, thereby performing one or more processes, including one or more of the processes described herein. Computer-executable instructions may be compiled or interpreted from computer programs created using a variety of programming languages and/or technologies including, without limitation, and either alone or in combination, Java, C, C++, C#, Fortran, Pascal, Visual Basic, Java Script, Perl, PL/SQL, etc. Display 28 is also in communication with computer processor 22 via connection system 16. Smart device 12 also optionally includes various in/out ports 30 through which data from a pointing device may be accessed by computer processor 22. Examples for the electronic devices include, but are not limited to, desktop computers, smart phones, tablets, or tablet computers.

Additional details of the invention are found in attached Exhibit A.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

Burman, B., & Margolin, G. (1992). Analysis of the association between marital relationships and health problems: An interactional perspective. Psychological Bulletin, 112, 39-63. doi: 10.1037/0033-2909.112.1.39

Cicila, L. N., Georgia, E. J., & Doss, B. D. (2014). Incorporating internet-based interventions into couple therapy: Available resources and recommended uses. The Australian and New Zealand Journal of Family Therapy, 35, 414. doi: 10.1002/anzf.1077

Coan, J. A., Schaefer, H. S., & Davidson, R. J. (2006). Lending a hand: Social regulation of the neural response to threat. Psychological Science, 17, 1032-1039. doi: 10.1111/j.1467-9280.2006.01832.x

Doss, B. D., Bensen, L. A., Georgia, E. J., & Christensen, A. (2013). Translation of Integrative Behavioral Couple Therapy to a web-based intervention. Family Process, 52, 139-153. doi: 10.1111/famp.12020

Gideon, J., Provost, E. M., & McInnis, M. (2016). Mood state prediction from speech of varying acoustic quality for individuals with bipolar disorder. Proceedings of the IEEE International Conference on Acoustics, Speech and Signal Processing, 2359-2363.

Grewen, K. M., Andersen, B. J., Girdler, S. S., & Light, K. C. (2003). Warm partner contact is related to lower cardiovascular reactivity. Behavioral Medicine, 29, 123-130. doi: 10.1080/08964280309596065

Hung, H., & Englebienne, G. (2013). Systematic evaluation of social behavior modeling with a single accelerometer. Processing of the ACM International Joint Conference on Pervasive and Ubiquitous Computing, 127-139. doi: 10.1145/2494091.2494130

Holt-Lunstad, J., Smith, T. B., & Layton, J. B. (2010). Social relationships and mortality risk: A meta-analytic review. PLoS Medicine, 7, e1000316. doi: 10.1371/journal.pmed.1000316

Ianakieva, I., Fergus, K., Ahmad, S., Pos, A., & Pereira, A. (2016). A model of engagement promotion in a professionally facilitated online intervention for couples affected by breast cancer. Journal of Marital and Family Therapy, 42, 701-715. doi: 10.1111/jmft.12172

Kim, S., Valente, F., & Vinciarelli, A. (2012). Automatic detection of conflict in spoken conversations: Ratings and analysis of broadcast political debates. Proceedings of IEEE International Conference on Acoustic, Speech, and Signal Processing, 5089-5092. doi: 10.1109/ICASSP.2012.6289065

Lane, N. D., Lin, M., Mohammod, M., Yang, X., Lu, H., Cardone, G., . . . & Choudhury, T. (2014). Bewell: Sensing sleep, physical activities and social interactions to promote wellbeing. Mobile Networks and Applications, 19, 345-359. doi: 10.1007/s11036-013-0484-5

Larson, J. H., Vatter, R. S., Galbraith, R. C., Holman, T. B., & Stahmann, R. F. (2007). The RELATionship Evaluation (RELATE) with therapist-assisted interpretation: Short-term effects on premarital relationships. Journal of Marital and Family Therapy, 33, 364-374. doi: 10.1111/j.1752-0606.2007.00036.x

Lawler, J. (2010). The real cost of workplace conflict. Entrepreneur. Accessed from https://www.entrepreneur.com/article/207196.

Leach, L. S., Butterworth, P., Olesen, S. C., & Mackinnon, A. (2013). Relationship quality and levels of depression and anxiety in a large population-based survey. Social Psychiatry and Psychiatric Epidemiology, 48, 417-425. doi: 10.1007/s00127-012-0559-9

Lee, Y., Min, C., Hwang, C., Lee, J., Hwang, I., Ju, Y., . . . Song J. (2013). SocioPhone: Everyday face-to-face interaction monitoring platform using multi-phone sensor fusion. Proceedings of the International Conference on Mobile Systems, Applications, and Services, 375-388. doi: 10.1145/2462456.2465426

Lunbald, A., & Hansson, K. G. (2005). The effectiveness of couple therapy: Pre- and post-assessment of dyadic adjustment and family climate. Journal of Couple & Relationship Therapy, 4, 39-55. doi: 10.1300/J398v04n04_03

Mayberry, R. M., Nicewander, D. A., Qin, H., & Ballard, D. J. (2006). Improving quality and reducing inequality: A challenge in achieving best care. Proceedings of Baylor University Medical Center, 19, 103-118.

Robles, T. F., & Kiecolt-Glaser, J. K. (2003). The physiology of marriage: Pathways to health. Physiology and Behavior, 79, 409-416. doi: 10.1016/50031-9384(03)00160-4

Sacker, A. (2013). Mental health and social relationships. Evidence Briefing of the Economic and Social Research Council. Accessed from http://www.esrc.ac.uk/files/news-events-and-publications/evidence-briefings/mental-health-and-social-relationships/

Sonnentag, S., Unger, D., & Nagel, I. (2013). Workplace conflict and employee well-being: The moderating role of detachment from work during off-job time. International Journal of Conflict Management, 24, 166-183. doi: 10.1108/10444061311316780

Springer, K. W., Sheridan, J., Kuo, D., & Carnes, M. (2007). Long-term physical and mental health consequences of childhood physical abuse: Results from a large population-based sample of men and women. Child Abuse & Neglect, 31, 517-530. doi: 10.1016/j.chiabu.2007.01.003

Timmons, A. C., Chaspari, T., Han, S. C., Perrone, L., Narayanan, S., & Margolin, G. (2017). Using multimodal wearable technology to detect conflict among couples. IEEE Computer, 50, 50-59. doi:10.1109/MC.2017.83

1. A method comprising:
receiving data streams from a plurality of mobile smart devices in the possession of a plurality of users, the data streams recording information about users' daily lives; and
sending intervention signals to a user in response to data acquired from two or more individuals and interpreted with respect to user internal states, moods, emotions, predetermined behaviors, and interactions with other users.

2. The method of claim 1 wherein the intervention signals are determined by algorithmic signal processing and/or machine learning solutions such that the intervention signals are responsive, interactive, and adaptive to the users.

3. The method of claim 2 further comprising incorporating human expert knowledge into a determination of the intervention signals.

4. The method of claim 3 wherein human expert knowledge is integrated and includes prompts sent at random intervals and/or according to specific time schedules.

5. The method of claim 4 wherein reminders designed to help users reach their daily goals are sent.

6. The method of claim 5 wherein the reminders include as spending a certain amount of time together, achieving a certain ratio of positive to negative interactions, or having a certain amount of physical contact.

7. The method of claim 1 wherein sending of interventions triggered by algorithms that automatically detect and predict moods and events to send prompts to oneself or to other users in a social network.

8. The method of claim 7 wherein moods and events include risky behaviors, extreme emotions, and/or negative moods.

9. The method of claim 7 wherein the prompts include warning people that conflict or other events are likely to occur, prompting people to engage in relaxation exercises, take a break, give a compliment, or to do something nice for someone else.

10. The method of claim 7 wherein the interventions also include sending prompts after events of interest have occurred.

11. The method of claim 7 wherein the prompts instruct users to reflect on an occurrence of an event, engage in relationship building activities, initiate positive contact, or discuss a topic together.

12. The method claim 1 further comprises providing feedback to the users to encourage beneficial aspects of interpersonal relationships.

13. The method of claim 12 wherein expert-knowledge is applied with personal and interpersonal information captured from human monitoring systems integrated through signal processing, data-scientific, and machine learning solutions.

14. The method of claim 13 wherein a human state is recognized, understood, and predicted and actionable feedback is provided to improve it in relation to corresponding relationship functioning.

15. The method of claim 12 wherein measurable indices of individual and interpersonal behavior consisting of input for closed-loop systems that automatically provide suggestions towards a desired state.

16. The method of claim 1 wherein heuristic, machine-learning, or control-theoretical approaches are applied and are automatically trained/tuned/perturbed towards optimizing a desired criterion to minimize conflict and maximize positive interactions.

17. The method of claim 1 wherein a model is constructed for interpersonal dynamics that occur when a set of individuals linked through a relationship interact with each other and with their environment.

18. The method of claim 1 further comprising learning each user's patterns over time so that accuracy and effectiveness of interventions increase with use.

19. The method of claim 1 further comprising investigating an impact of each prompt and intervention on individual and interpersonal functioning and providing feedback about which interventions are most helpful population-wide and which are better for specific users or groups of users.

20. The method of claim 1 wherein intervention schemes are performed quantitatively through signal- and data-derived measures indicative of individual characteristics and relationship functioning concepts.

21-23. (canceled)
